(19) United States
(12) Patent Application Publication
(10) Pub. No.: US 2015/0320576 A1
(43) Pub. Date: Nov. 12, 2015

(54) APPARATUS AND METHOD FOR SUSPENDING RESIDUUM PROTECTION DEVICE

(71) Applicants: Laura Brewer Riedel, Durham, NC (US); Michael Joseph Astilla, Durham, NC (US)

(72) Inventors: Laura Brewer Riedel, Durham, NC (US); Michael Joseph Astilla, Durham, NC (US)

(21) Appl. No.: 14/706,537

(22) Filed: May 7, 2015

Related U.S. Application Data
(60) Provisional application No. 61/989,811, filed on May 7, 2014.

(51) Int. Cl. A61F 2/78

(52) U.S. Cl. CPC A61F 2/7812; A61F 2/78; A61F 2002/7881; A61F 2002/7862; A61F 2002/7837

(57) ABSTRACT

An apparatus is provided for supporting a post amputation residuum protection device. The supporting apparatus comprises a central portion, and a first flap and a second flap extending from the lower end of the central portion. A strap member is secured to the central portion and includes a pair of elongated straps each having a free distal end and longitudinally spaced holes adjacent their distal ends. The flaps are adapted to extend around a distal end of the residuum protection device. The first side and the second side of the central portion are adapted to wrap around the residuum protection device forming a shape conforming to the outside of the residuum protection device. The straps are connectable to a waist belt for supporting the residuum protection device.

### CROSS-REFERENCES

This application is related to U.S. provisional application No. 61/989,811, filed May 7, 2014, entitled “SUSPENSION DEVICE FOR POST AMPUTATION RESIDUUM PROTECTION DEVICES”, naming Laura Brewer Riedel and Michael Joseph Astilla as the inventors. The contents of the provisional application are incorporated herein by reference in their entirety, and the benefit of the filing date of the provisional application is hereby claimed for all purposes that are legally served by such claim for the benefit of the filing date.

### BACKGROUND

An apparatus is described for supporting a post amputation residuum protection device.

A post amputation residuum protection device provides for post amputation residuum maintenance. The residuum protection device stabilizes an amputation residuum while accommodating fluctuations in the volume and the diameter of the residuum. A known residuum device includes a posterior shell, a pre-tibial shell, optional enclosure panels, and a circular pad. The pre-tibial shell is flexible and movable within the posterior shell for changes in limb volume and diameter variance.

Post amputation residuum protection devices require a method for suspending the device to ensure that they do not migrate away from the residuum. Conventionally, a fork strap suspended from a belt has been secured to the device. The fork strap is connected to the device with rivets or screws or via anatomical suspension on the residuum. However, manufacture of the fork strap and belt are labor intensive and time consuming. In addition, anatomical suspension requires remaking the complete device as the residuum anatomy changes (e.g. volume fluctuation). As a result, these techniques often result in sub-optimal suspension and residuum protection.

For the foregoing reasons, there is a need for an improved apparatus and method for suspending a post amputation residuum protection device. The improved apparatus and method should ideally prevent the device from migrating away from the residuum.

### SUMMARY

An apparatus is provided for supporting a post amputation residuum protection device having a distal end. The supporting apparatus comprises a central portion having a longitudinal axis, an upper end, a lower end, a first side extending laterally in a first direction from the longitudinal axis, and a second side extending laterally in a second direction from the longitudinal axis, the second direction opposite the first direction. A first flap extends from the lower end of the central portion, and a second flap extends from the lower end of the central portion spaced from the first flap. A strap member is secured at a proximal end to the central portion and includes a pair of elongated straps each having a free distal end, the pair of straps having longitudinally spaced holes adjacent their distal ends. Each of the central portion, first flap and second flap have an inner surface adapted to be proximate to the residuum protection device and an outer surface adapted to face away from the residuum protection device. The flaps are adapted to extend around the distal end of the residuum protection device. The first side and the second side of the central portion are adapted to wrap around the residuum protection device forming a shape conforming to the outside of the residuum protection device.

A method is provided for supporting a post amputation residuum including a distal end. The supporting method comprises providing a wrap. The wrap includes a central portion having a longitudinal axis, an upper end, a lower end, a first side extending laterally in a first direction from the longitudinal axis, and a second side extending laterally in a second direction from the longitudinal axis, the second direction opposite the first direction. A first flap extends from the lower end of the central portion, and a second flap extends from the lower end of the central portion spaced from the first flap. A strap member is secured at a proximal end to the central portion and includes a pair of elongated straps each having a free distal end. The pair of straps have longitudinally spaced holes adjacent the distal ends. Each of the central portion, first flap and second flap have an inner surface adapted to be proximate to the residuum protection device. The method further comprises the steps of extending the first flap and the second flap around the lower distal end of the residuum protection device, and overlapping the first side and the second side of the central portion around the residuum protection device forming a shape conforming to the outside of the residuum protection device.

### BRIEF DESCRIPTION OF THE DRAWINGS

FIG. 1 is a top perspective view of an embodiment of an apparatus for suspending a residuum protection device.

FIG. 2 is a top plan view of the suspending apparatus as shown in FIG. 1.

FIG. 3 is a bottom plan view of the suspending apparatus as shown in FIG. 1.

FIG. 4 is a top side elevation view of the suspending apparatus as shown in FIG. 1.

FIG. 5 is a bottom side plan view of the suspending apparatus as shown in FIG. 1.

FIG. 6 is a left side elevation view of the suspending apparatus as shown in FIG. 1.

FIG. 7 is a right side elevation view of the suspending apparatus as shown in FIG. 1.

FIG. 8 is a front perspective view of the suspending apparatus as shown in FIG. 1 assembled with a belt and strap.

FIG. 9 is an exploded front perspective view of the suspending apparatus as shown in FIG. 8.

FIGS. 10-13 are perspective views of the suspending apparatus as shown in FIG. 1 showing the steps for applying the suspending apparatus to a residuum protection device.

### DESCRIPTION

Certain terminology is used herein for convenience only and is not to be taken as a limitation on the invention. For example, words such as “upper,” “lower,” “left,” “right,” “horizontal,” “vertical,” “upward,” and “downward” merely describe the configuration shown in the FIGs. Indeed, the components may be oriented in any direction and the terminology, therefore, should be understood as encompassing such variations unless specified otherwise. Throughout this disclosure, where a process or method is shown or described, the steps of the method may be performed in any order or simultaneously, unless it is clear from the context that one step depends on another being performed first.

The term “residuum” as used herein includes the remaining portion of an amputated body part, such as an amputated limb and, in particular, a leg. When used with a limb, such as a leg, the residuum device helps with knee range of motion preservation.

It is understood that, although the suspension apparatus will be described in detail herein with reference to a particular post-amputation residuum device as described in U.S. Pat. No. 8,679,193, the contents of which are incorporated by reference herein in their entirety, the suspension apparatus may be applied to, and find utility with, residuum devices. As described above, there a number of devices used for post amputation residuum protection. Further, although the present suspending apparatus will be described in detail herein as embodied for use with a particular device, it is not intended to be so limited. The present apparatus may be used in other devices. Thus, the suspending apparatus has general applicability to any device providing residuum protection wherein improvements in suspension and ease of use are desired.

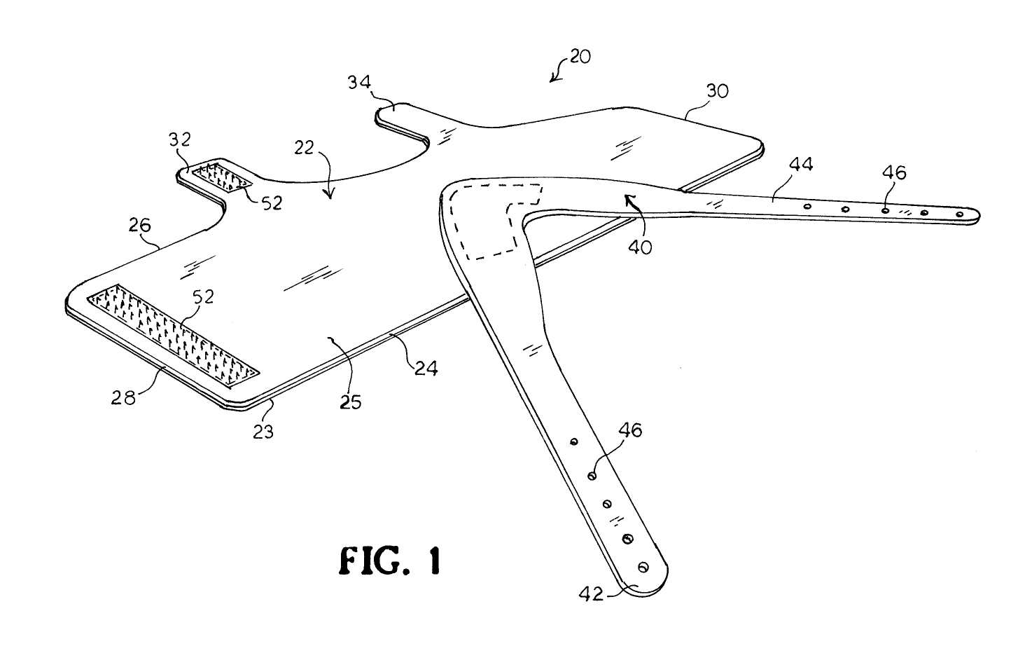

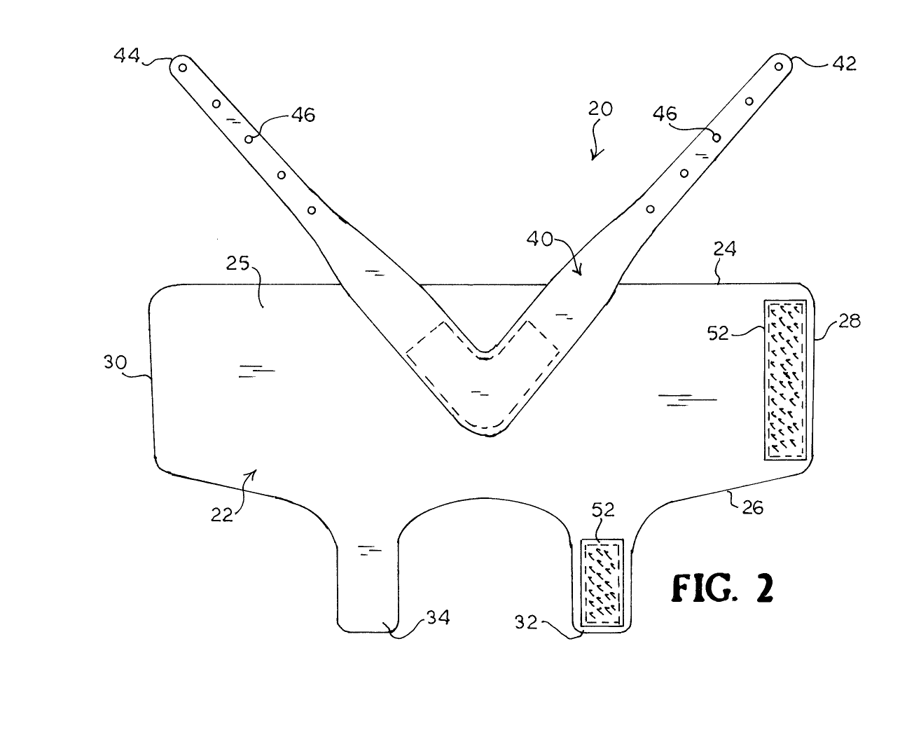

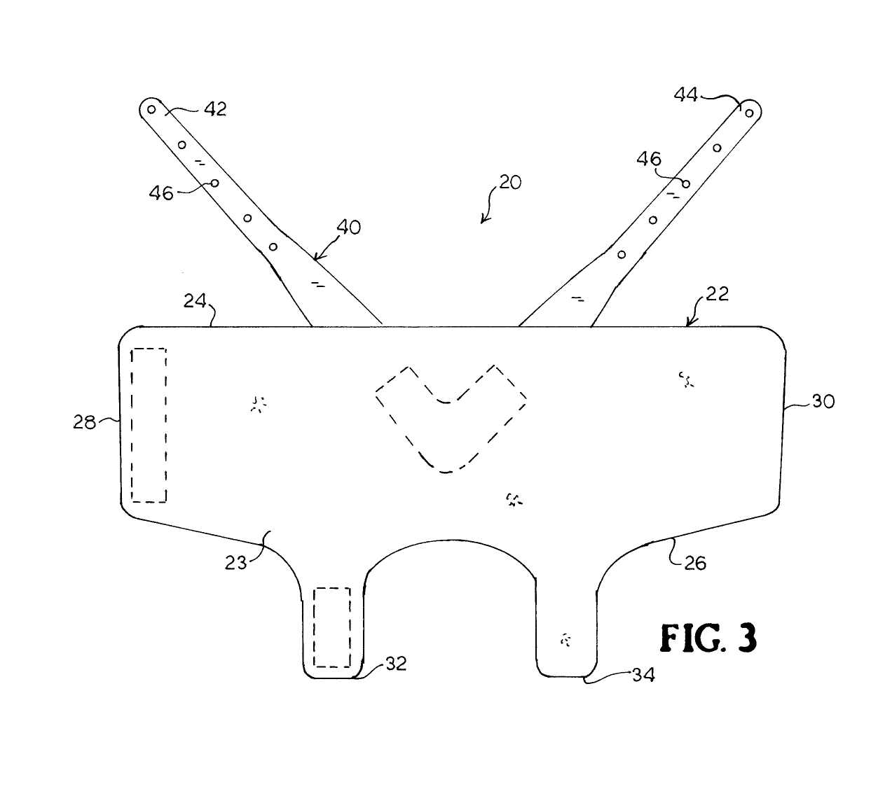

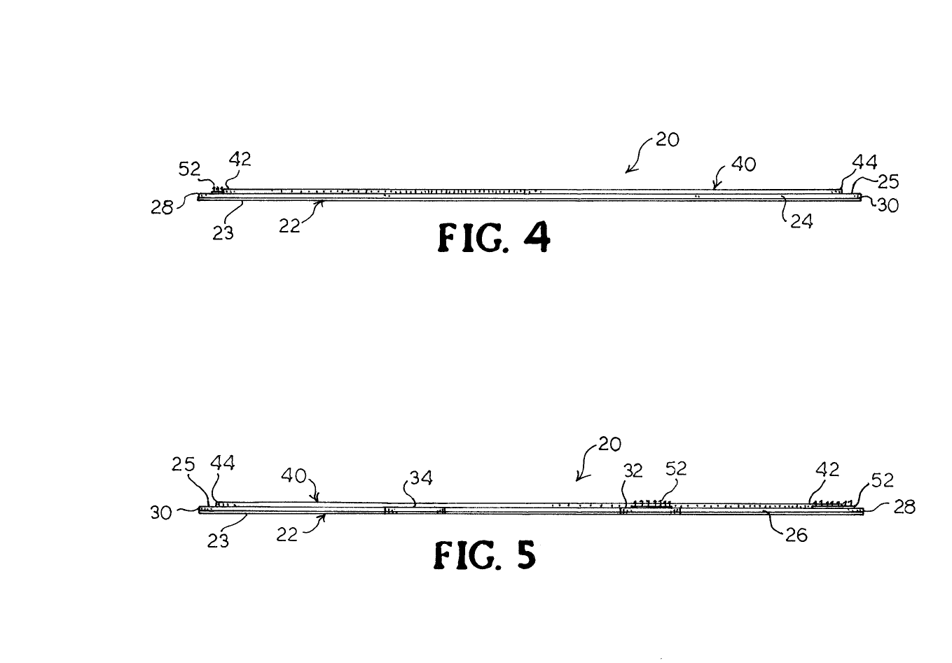

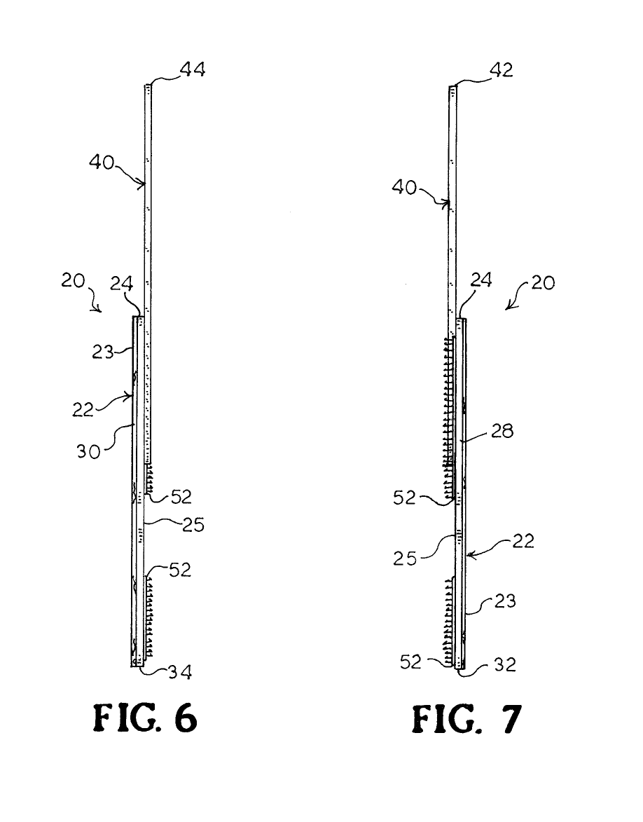

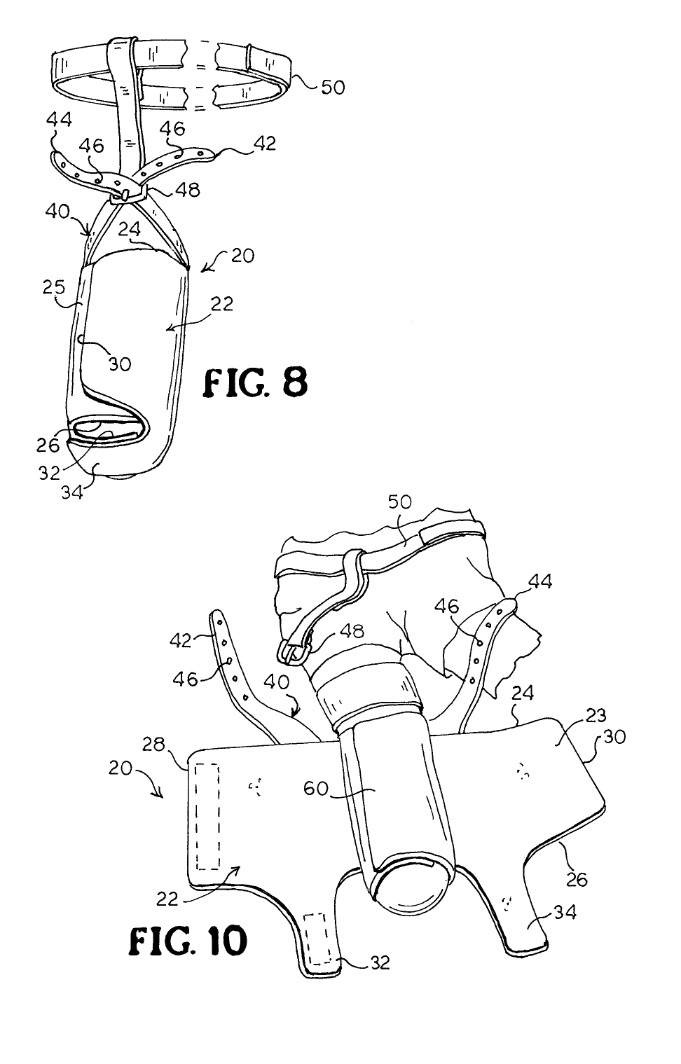

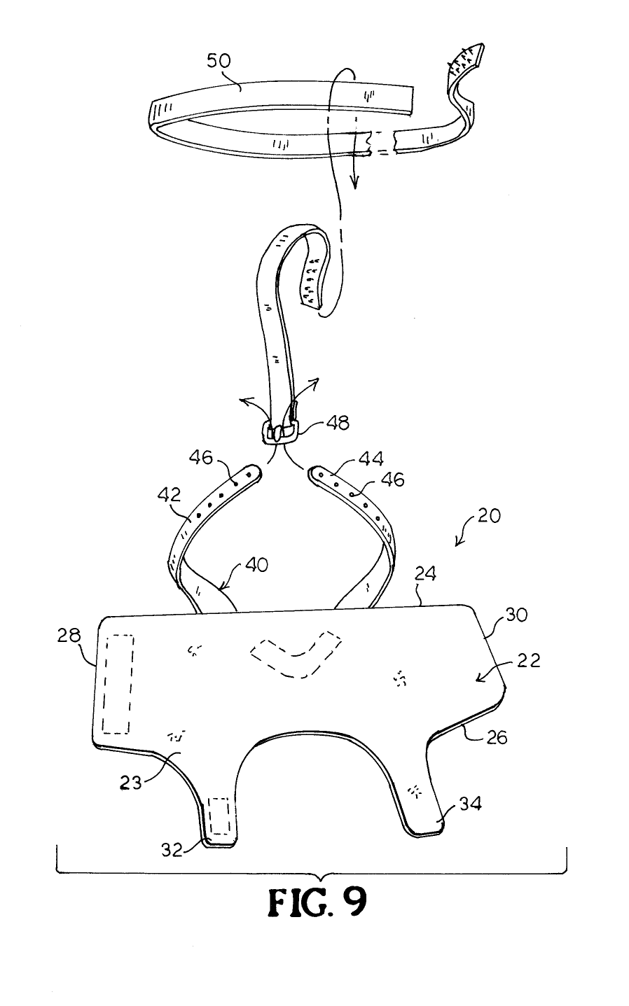

Referring now to the drawings, wherein like reference numerals designate corresponding or similar elements throughout the several views, an embodiment of an apparatus for suspending an amputation residuum device is shown in FIGS. 1-7 and generally designated at 20. The outer surface 25 of the suspending apparatus 20 is shown in FIG. 2, and the inner surface 23 of the suspending apparatus 20 is shown in FIG. 3. When worn by a user, the inner surface 23 of the suspending apparatus 20 is adjacent to the residuum, while the outer surface 25 faces away from the user's body.

The suspending apparatus 20 comprises a central portion 22 having a longitudinal axis A-A, an upper end 24 and an opposite lower end 26, a first side 28 extending laterally in a first direction from the longitudinal axis, and a second side 30 extending laterally in a second direction from the longitudinal axis in a direction opposite the first direction. A first flap 32 extends from the lower end 26 of the first side 28 of the central portion 22, and a second flap 34 extends from the lower end 26 of the second side 30 of the central portion 22. Both flaps 32, 34 extend generally parallel to the longitudinal axis of the central portion 22. Each flap 32, 34 has an inside surface adapted to contact the residuum protection device, an outside surface adapted to face away from the residuum protection device, and a free end. The first flap 32 and the second flap 34 are configured to extend across a lower end of an amputation residuum protection device and fasten to each other with hook and loop fasteners. Similarly, the central portion 22 is configured to extend around the residuum protection device and the first side 28 and the second side 30 fasten to each other such that the central portion 22 forms a sleeve-shape conforming to the outside of the residuum protection device for supporting the residuum protection device.

A V-shaped strap member 40 is attached to the central portion 22 such that the midpoint of the “V” runs substantially along the longitudinal axis A-A of the central portion. An elongated right strap 42 and an elongated left strap 44 extend to free distal ends from the central portion 22 at an angle with respect to the longitudinal axis A-A. The straps 42, 44 have longitudinally spaced holes 46 along their length. The straps 42, 44 are configured to extend at least partially around the sleeve-shaped central portion 22 and overlap. The straps 42, 44 are fastened to a buckle 48 suspended from a belt 50 worn by the user. The straps 42, 44 are joined to the buckle 48 where a hole from each of the intersecting straps 42, 44 intersect. Any other of the holes 46 may be selected to further tighten or loosen the tension on the suspending apparatus 20 to provide support to the residuum protection device.

In the embodiment shown, the central portion 22, first and second flaps 32, 34, and the first and second straps 42, 44 may be made of a one piece of material, with two layers sewn together continuously along the periphery. The material used for the suspending apparatus 20 comprises elastic neoprene and may have a composition that includes spandex. The remainder of the material may be, or include, for example, cotton and polyester. Other fabrics and combinations of materials may be used as recognized by one of ordinary skill in the art, so long as the material exhibits a degree of elasticity that provides an expandable and flexible, yet snug, fit with pressure that may be adequate to provide support of the residuum protection device.

The strap member 40 may likewise be made of two or more layers of the same material. The strap member 40 is sewn to the central portion 22 at the intersection of the straps 42, 44. In the FIGs., dashed lines on the suspending apparatus 20 indicate stitching for attachments of the parts, such as the strap member 40. One alternative configuration to the strap member 40 may include the use of fabric straps, webbed straps, or elastic bands with clips or other connection means.

Means are provided for operatively connecting together the first side 28 and the second side 30 of the central portion 22 to form a sleeve around the residuum protection device. In some embodiments, mating fasteners are provided on the outer surface of the first side 28 and the inner surface of the second side 30, and mating fasteners are provided on the inner surface of the first flap 32 and the outer surface of the second flap 34. In the embodiments shown herein, hook and loop fasteners, such as Velcro.®. (VELCRO is a registered trademark of Velcro Industries B.V.), are used to removably attach the parts of the suspending apparatus 20 to each other. The hook material is designated at 52 and the loop material is designated at 54, but the position of the materials may be switched so long as a hook surface meets a loop surface when the material is brought together. In the embodiments shown, there are hook sections 52 of fasteners on the outer surface of the first side 28 and the first flap 32. The material of the inner surface of the second side 30 and the second flap 34 functions as loop fasteners 54 such that the sides 28, 30 and the flaps 32, 34 may be connected at any point of contact on their inner surface. Other fasteners may be used as recognized by one of ordinary skill in the art, such as a series of buttons or snaps, or a combination of webbed straps and clips.

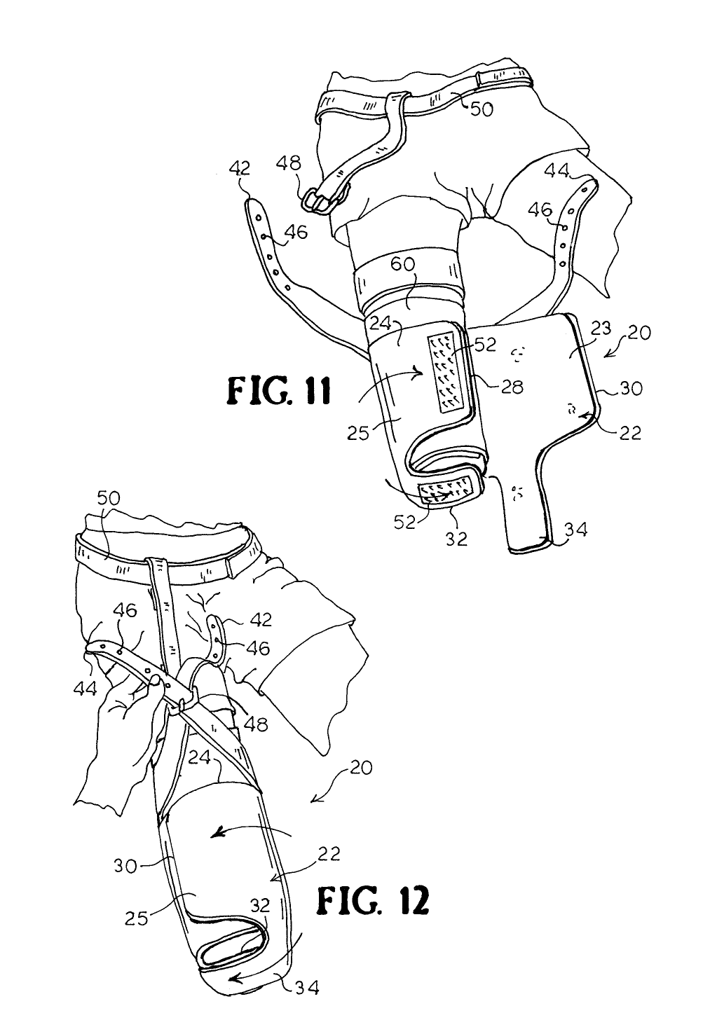

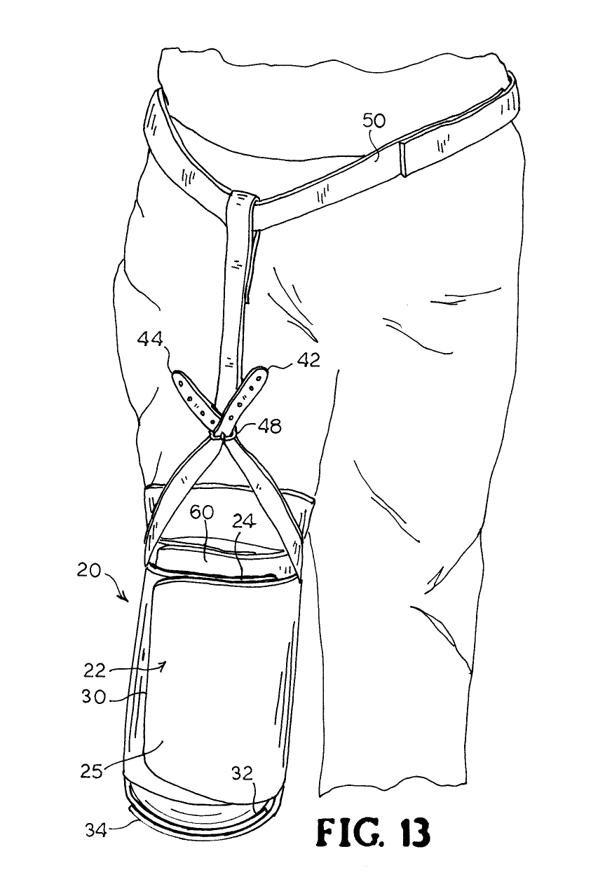

In use, the suspending apparatus 20 may be applied to a residuum protection device 20 as shown in FIGS. 10-13. First, as shown in FIG. 10, the inner surface of the central portion 22 is placed against the back of the residuum protection device 60 with the lower end 26 of the suspending apparatus 20 proximate to the lower end of the residuum and the longitudinal axis of the suspending apparatus 20 aligned with the length of the residuum. Next, the first flap 32 and the second flap 34 are crossed under the lower distal end of the residuum protection device 60. The inner surface of the second flap 34 passes over the top of the first flap 32 and the flaps are secured to each other with fasteners to achieve a snug fit. The secured flaps 32, 34 provide compressive resistance to distal migration of the residuum protection device 60. The first side 28 of the central portion 22 is wrapped at least partially around the residuum protection device 60. The second side 30 of the central portion 22 is then wrapped around the residuum protection device 60 to at least partially overlap the first side 28. The inner surface of the second side 30 is pressed into contact with the outer surface of the first side 28 to secure the sides together such that the suspending apparatus 20 encloses the residuum protection device 60 and applies pressure to the device.

The user wraps the straps 42, 44 from posterior of the residuum to anterior at the height of the tibia along the medial and lateral sides of the patella. The strap members are configured to extend from their proximal end at the connection to the central portion 22 and around the sleeve formed by the central portion 22. The straps 42, 44 are overlapped at the height of the femur. Holes 46 from each strap 42, 44 are aligned at an appropriate location and fastened to the suspended buckle 48 at the anterior of the thigh of the user. The trajectory of the straps 42, 44 tracks along the medial and lateral aspects of the patella for controlling internal and external rotation of the residuum protection device 60 on the limb while encouraging a neutral knee flexion angle. The holes 46 used may be selected to appropriately adjust the tension on the belt 50 for supporting the suspending apparatus 20. The selected holes 46 are determined by such variables as overall length of the residuum, femur length, and range of motion deficits. The buckle 48 is attached to the waist belt 50 via a strap with the appropriate tension to ensure proper suspension of the residuum protection device 60. If desired, the straps 42, 44 may be left attached when removing the suspending apparatus 20 to save the step of fastening the straps 42, 44 the next time the suspending apparatus 20 is put on the user.

The suspending apparatus has many advantages, including a flexible wrap-around design configured to accommodate the size and shape of the amputation resdiuum protection device and provide a secure and comfortable fit. The one size design requires less inventory and no screws or rivets for attachment. The wrap around design enhances post amputation protection device suspension and adjustability. Further, it will be understood that the suspending apparatus 20 may be used with or without the residuum protection device 60 to protect a residuum, therapy or soft dressing containment, depending on the needs or condition of the user.

Although the suspending apparatus has been shown and described in considerable detail with respect to only a few exemplary embodiments thereof, it should be understood by those skilled in the art that we do not intend to limit the suspending apparatus to the embodiments since various modifications, omissions and additions may be made to the disclosed embodiments without materially departing from the novel teachings and advantages of the suspending apparatus, particularly in light of the foregoing teachings. Accordingly, we intend to cover all such modifications, omissions, additions and equivalents as may be included within the spirit and scope of the suspending apparatus as defined by the following claims. In the claims, means-plus-function clauses are intended to cover the structures described herein as performing the recited function and not only structural equivalents but also equivalent structures. Thus, although a nail and a screw may not be structural equivalents in that a nail employs a cylindrical surface to secure wooden parts together, whereas a screw employs a helical surface, in the environment of fastening wooden parts, a nail and a screw may be equivalent structures.

We claim:

1. An apparatus for supporting a post amputation residuum protection device having a distal end, the supporting apparatus comprising:
a central portion having a longitudinal axis, an upper end, a lower end, a first side extending laterally in a first direction from the longitudinal axis, and a second side extending laterally in a second direction from the longitudinal axis, the second direction opposite the first direction;
a first flap extending from the lower end of the central portion;
a second flap extending from the lower end of the central portion spaced from the first flap; and
a strap member secured at a proximal end to the central portion and including a pair of elongated straps each having a free distal end, the pair of straps having longitudinally spaced holes adjacent the distal ends,
wherein each of the central portion, first flap and second flap have an inner surface adapted to be proximate to the residuum protection device and an outer surface adapted to face away from the residuum protection device, and wherein the flaps are adapted to extend around the distal end of the residuum protection device, and the first side and the second side of the central portion are adapted to wrap around the residuum protection device forming a shape conforming to the outside of the residuum protection device.

2. The supporting apparatus of claim 1, wherein each of the straps is configured to wrap in opposite directions around the wrapped central portion and overlap one another adjacent to the upper end of the central portion such that a hole of each strap is aligned, and further comprising a connector disposed in the aligned holes for securing the straps together.

3. The supporting apparatus of claim 1, further comprising a waist belt operatively attached to the connector.

4. The supporting apparatus of claim 1, wherein the first side and the second side are configured to fasten together to form a sleeve around the residuum protection device.

5. The supporting apparatus of claim 1, further comprising mating fasteners on the outer surface of the first side and the inner surface of the second side, and mating fasteners on the inner surface of the second flap and the outer surface of the first flap.

6. The supporting apparatus of claim 5, wherein the mating fasteners comprise hook and loop fasteners.

7. A method for supporting a post amputation residuum including a distal end, the supporting method comprising:
providing a wrap including
a central portion having a longitudinal axis, an upper end, a lower end, a first side extending laterally in a first direction from the longitudinal axis, and a second side extending laterally in a second direction from the longitudinal axis, the second direction opposite the first direction,
a first flap extending from the lower end of the central portion,
a second flap extending from the lower end of the central portion spaced from the first flap, and
a strap member secured at a proximal end to the central portion and including a pair of elongated straps each having a free distal end, the pair of straps having longitudinally spaced holes adjacent the distal ends, wherein each of the central portion, first flap and second flap have an inner surface adapted to be proximate to the residuum protection device;
extending the first flap and the second flap around the lower distal end of the residuum protection device; and
overlapping the first side and the second side of the central portion around the residuum protection device forming a shape conforming to the outside of the residuum protection device.

8. The supporting method of claim 7, further comprising the step of wrapping each of the pair of straps in opposite directions around the central portion and overlapping the free distal ends.

9. The supporting method of claim 8, further comprising the step of providing a connector configured to be received in aligned holes of the overlapped straps for securing the straps together.

10. The supporting method of claim 9, further comprising the step of providing a waist belt worn by a user, and operatively attaching the belt to the connector for supporting the wrap.

11. The supporting method of claim 9, further comprising the step of changing the position of the connector in the aligned holes along the straps, wherein the tension of the supporting apparatus is adjusted.
